(19) United States
(12) Patent Application Publication
(10) Pub. No.: US 2005/0276761 A1
(43) Pub. Date: Dec. 15, 2005

(54) Zeolite based UV Absorbing and Sunscreen Compositions

(76) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(21) Appl. No.: 10/710,011

(22) Filed: Jun. 11, 2004

(51) Int. Cl. A61K 7/42

(52) U.S. Cl. 424/59

(57) ABSTRACT

The present invention discloses novel applications of zinc zeolite and titanium zeolite in UV absorbing and free radical neutralizing compositions. Additionally, the preparation of such metal zeolites by a simple in-situ process is also disclosed. This process is useful for the preparation of zinc zeolite and titanium zeolite compositions either in a hydrous or in an anhydrous form. These compositions are useful for topical sunscreen and skin protection applications.

### CROSS REFERENCE TO RELATED APPLICATIONS

Certain Zeolite derivatives in an unrelated application have been disclosed in U.S. patent application Ser. No. 10/605,191 (Baby Care Skin Protectant Compositions for Diaper Rash. Filed Sep. 13, 2003).

### BACKGROUND OF INVENTION

For products desirable for topical delivery, there is usually a belief that the faster the absorption of such compositions into the skin the better they are. Although that may be applicable to certain compositions it is not universally desirable. For example, the protection of skin surface from factors such as UV and free radicals requires that such protective compositions be delivered and allowed to remain on the skin surface for as long as necessary to provide their maximum topical benefits.

Inorganic UV absorbers such as zinc oxide and titanium dioxide have been used for a long time in various sunscreen protection compositions. One of the greatest disadvantage of such inorganic sunscreens is that such compositions cause unwanted excessive temporary whitening of skin. Such sunscreen compositions, when applied to nose or face, for example, make nose or face look chalky white and unattractive. A number of micronized forms of both zinc oxide and titanium dioxide have been produced commercially to circumvent this difficulty. Although such micronized forms of zinc oxide and titanium dioxide are known to cause less skin whitening effect, the skin whitening is still unacceptable to many consumers.

The present invention discloses a surprising and unexpected discovery that zinc zeolites and titanium zeolites provide excellent protection from UV and free radicals when applied topically similar to a sunscreen product. Moreover, these zeolite derivatives do not cause excessive skin whitening effect in the same manner as caused by zinc oxide and titanium dioxide. Additionally, the surface area of zinc or titanium ions is increased so that smaller amounts of these compositions can be used to provide UV and free radical neutralization benefits compared to zinc oxide and titanium dioxide.

Zeolites are a group of crystalline aluminosilicates that have a porous structure with a cavity. The preparation and properties of these zeolites are described in detail in U.S. Pat. No. 2,882,243, among other sources. Generally, the preparation involves combining aqueous solutions that are sources of silica, alumina and sodium to produce a gel which crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300° C. provides anhydrous zeolite.

Zeolites have the following properties that can be highly useful for topical delivery of cosmetic and pharmaceutical compositions: (1) Zeolites have high adsorptive capacity for water and many organic compounds including toxic metals and enzymes (which makes them useful for many other applications such as water purification, waster water treatment, and chemicals refining/purification), (2) Zeolites are available in certain pore sizes that can be used for self-warming or non-warming cosmetic and pharmaceutical compositions, (3) Zeolites can be made anionic or cationic, which can be used for controlled-release of certain cosmetic and pharmaceutical ingredients via ion-pair mechanisms, (4) Zeolites have a very large surface area that can nearly achieve a nano-particle distribution of organic molecules attached to its vast surface area, (5) Zeolites can also be made in cations other than sodium or potassium, (6) Zeolites do not absorb into the skin, which is useful for topical delivery of cosmetic and pharmaceutical compositions that are electronically attached to such zeolite surfaces for their controlled or slow delivery over a period of time, and (7) Zeolites cause much less skin whitening effect compared to other inorganic materials such as zinc oxide and titanium dioxide.

However, many of the prior art applications of zeolites have centered upon their chemical catalysis, heat releasing, or trapping of small molecular weight ingredients. Zeolites also have outer surface area, in addition to such inner pore surface areas. The functional properties of zeolites utilizing both inner and outer surface areas have not been disclosed in the prior art, as shall become evident further.

Zeolites can be made with both specific pore structures and bound cations that have found applications in various self-warming cosmetic compositions. U.S. Pat. No. 3,250,680 (Menkart et al.) discloses applications of Zeolites for the preparation of self-heating toothpaste and other such compositions. This utilizes only the heat releasing property of zeolites.

U.S. Pat. No. 4,626,550 (Hertzenberg) discloses certain personal care products such as lotions and creams that are prepared using Zeolite A that contains sodium and potassium.

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various problems.

U.S. Pat. No. 6,274,128 (Bergman et al.) discloses an essentially anhydrous hair conditioning composition comprising zeolites of specific pore size larger than the critical diameter of a water molecule and both the carrier molecules and the hair conditioner molecules that have molecular diameters larger than the largest average pore size of the micro porous materials. As is clearly evident, such constraints are not convenient or commercially achievable at a reasonable cost.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect.

U.S. Application 20010016201 (Janchitraponvej) discloses a yet another self-heating application of an anhydrous rinse-out hair care composition utilizing zeolites.

Zeolites can also be made in cations other than sodium or potassium. U.S. Patent Application 20040014591 (Muller et al.) discloses titanium and other transition metal zeolites. U.S. Patent Application 20010021368 (Masini et al.) discloses certain metal zeolites, including zinc zeolite. U.S. Pat. No. 6,357,678 (Hu et al.) discloses preparation of zinc zeolites by a very difficult multi-step process. U.S. Pat. No. 6,605,267 (Lee et al.) discloses process for making metal zeolites with quaternary ammonium compounds, useful as chemical catalysts. U.S. Pat. No. 6,084,142 (Yao et al.) discloses the preparation of a zinc zeolite, and its application in petroleum cracking process. U.S. Pat. No. 6,177,374 (Pradhan et al.) discloses the preparation of silicon, zinc and aluminum zeolites, and their application in petroleum cracking process. Yao and Pradhan do not disclose any cosmetic or UV absorber applications of such zeolite derivatives. U.S. Pat. No. 6,479,427 (Anthony at al.) and U.S. Pat. No. 5,502,240 (Pugach) disclose titanium zeolites and their application in petroleum cracking process. U.S. Pat. No. 5,772,917 (Kynast et al.) discloses a cesium zeolite that is luminescent. U.S. Pat. No. 6,106,797 (Muller et al.) discloses titanium or vanadium zeolites useful for accelerating oxidation reactions. U.S. Pat. No. 6,008,389 (Grosch et al.) discloses titanium and vanadium zeolites useful as catalysts for the preparation of epoxides, in particular propylene oxide, from olefins, hydrogen and oxygen. U.S. Patent Application 20030035763 (Vergani et al.) discloses the use of iron and manganese zeolites in the purification of organometallic compounds utilizing such zeolite's adsorptive properties. U.S. Patent Application 20030024856 (Surana et al.) discloses a yet another application of zeolite's adsorptive properties in removing odors. U.S. Patent Application 20020127402 (Green et al.) discloses the antimicrobial applications of silver ions attached to zeolites by ion-exchange methods.

It is worthy of note that although zeolites with many different cations, such as titanium, zinc, manganese, iron, quaternary ammonium, and copper have been disclosed, any applications of such metal zeolites for UV absorption or free radical neutralization applications have not been disclosed. It is further worthy of note that both titanium and zinc are well known in their oxide state as sun block agents that have been used in sunscreen compositions now for several years. It is further worthy of note that zinc salts are known for their antimicrobial, skin protectant, and anti-irritant properties. Zinc oxide, for example, is a FDA-approved drug ingredient for skin protectant compositions. Zinc acetate, zinc chloride, zinc carbonate, zinc ricinoleate, and zinc sulfate have all been used for antiseptic, astringent, and skin protective compositions, as mentioned in The Merck Index, 12^{th} Edition (1996). The divalent salts of zinc, in general, have antibacterial and skin protectant properties.

The UV absorbing and free radical neutralizing benefits of zinc zeolite and titanium zeolite have not been reported in the prior art.

This lack of prior art knowledge is of special note, since zeolites with enhanced ion-exchange capacity are well known (U.S. Patent Application 20010053741, Mikko et al.; U.S. Pat. No. 5,935,891; Prior). U.S. Pat. No. 6,503,740 (Alther et al.) discloses zeolites treated with an organic modification compound such as quaternary amines, pyridinium compounds, and phosphonium amines that are useful for water treatment applications. U.S. Pat. No. 6,365,130 (Barry et al.) discloses zeolites exchanged with antimicrobial metals for a chewing gum application, or a laundry application (U.S. Pat. No. 6,454,813; Chan). Modified zeolites have been used for topical cancer therapy (U.S. Pat. No. 6,288,045; Kaufman). Additionally, U.S. Pat. No. 4,620,929 (Hofmann) and U.S. Pat. No. 3,935,067 (Thayer) teach the use of expanded clay or plastic materials in combination with bentonite, which advantageously exhibits moisture retention characteristics.

### SUMMARY OF INVENTION

The present invention discloses novel applications of zinc zeolite and titanium zeolite in UV absorbing and free radical neutralizing compositions. Additionally, the preparation of such metal zeolites by a simple in-situ process is also disclosed. This process is useful for the preparation of zinc zeolite and titanium zeolite compositions either in a hydrous or in an anhydrous form.

### DETAILED DESCRIPTION

Zinc and titanium derivatives, such as zinc oxide and titanium dioxide, have been used as UV absorber and sunscreen agents. It is clear that metal in such compositions is attached to at least one oxygen, such as in zinc oxide, or two oxygen atoms, such as in titanium dioxide. In the present invention, zinc is attached to two oxygen atoms and titanium may be attached to four oxygen atoms of the aluminosilicate moiety of zeolite backbone. It is hypothesized by the present inventor that it is the zinc or titanium metal cations that provide this UV absorbing efficacy, and the anion part of ion-pair, such as oxide, does not provide any significant biological role.

I have now found a very simple solution to this unwanted skin whitening problem caused by zinc oxide and titanium dioxide in UV absorbing and sunscreen topical applications by developing zinc or titanium zeolite derivatives in which zinc or titanium cations are attached to two or four oxygen atoms, respectively, which are then attached to a silicone atom, silicate, or aluminosilicate of a zeolite backbone. Such zinc or titanium aluminosilicates are easy to prepare, and they provide a topical source for zinc or titanium. These zinc or titanium zeolite compositions of the present invention are made from commonly available zinc or titanium salts, and zeolites. Since the diameter of divalent zinc cation is only 0.69 Angstrom units and tetravalent titanium cation is 0.68 Angstrom units, which are actually smaller than the Van der Waals radius of water molecule, 2.82 Angstrom units. Divalent zinc and tetravalent titanium cations can thus easily enter the cavity of zeolite of even the smallest pore size, which can be 3 to 4 Angstrom units.

Zeolites have a very large surface area that is ionic in its nature. This surface area covers both the outside of zeolite and the inside zeolite's porous cavity. The total surface area of a zeolite can be 300 square meters per gram of zeolite. The size of the pores of this cavity determines the size of any molecules that can enter zeolite's internal cavity.

The present invention also discloses simple in-situ preparation of divalent and polyvalent metal zeolites and their application in UV absorbing and free radical neutralizing compositions. The divalent and polyvalent metal zeolites can be prepared by a very simple process by the ion-pair exchange of a zinc or titanium derivative (such as zinc chloride, titanium tetrachloride, zinc sulfate, titanium sulfate, zinc nitrate, zinc acetate, zinc gluconate, titanium gluconate, zinc EDTA, etc.) with a zeolite, as illustrated in Equation 1, 2, and for the preparation of zinc zeolite and Equation 4 for the preparation of titanium zeolite.
Zinc Chloride+Zeolite→Zinc Zeolite+Sodium (potassium) Chloride (Equation 1).
Zinc Acetate+Zeolite→Zinc Zeolite+Sodium (potassium) Acetate (Equation 2).
Zinc Gluconate+Zeolite→Zinc Zeolite+Sodium (potassium) Gluconate (Equation 3).
Titanium Tetrachloride+Zeolite→Titanium Zeolite+Sodium (potassium) Chloride (Equation 4).

It should be noted that zeolites contain sodium and potassium cations that can be exchanged with other cations. It is commonly known that the exchange efficiency is in the following order for some metals: Ba>Pb>Cd>Zn>Cu>K>Na>Li. The exchange amount is determined by the exchange capacity of such zeolites, which is usually expressed as milli-equivalents (meq) of a cationic composition to per gram weight of zeolite. A zeolite with 1.0 meq per gram exchange capacity, for example, can exchange 0.068 grams of zinc chloride per gram of such zeolite. This is calculated as follows. The molecular weight of zinc chloride is 136.3. Thus, 136.3 grams of zinc chloride equals 1000 milli-equivalents (or, 1 mole equivalent), or 0.136 grams of zinc chloride equals one milli-equivalent. Since each zinc chloride molecule has two chlorine atoms that can undergo exchange, only half the equivalent amount of zinc chloride will thus be needed to exchange monovalent cations (such as sodium or potassium) in that zeolite. Thus, only 0.06815 grams of zinc chloride will be needed to exchange with one gram of zeolite for a complete exchange (i.e. 136.3/1000/2=0.06815). In practice, total exchange is not required. Typically, only 10 to 50% of all available monovalent cations need to be exchanged. In another example, 0.0917 grams of zinc acetate (molecular weight 183.4) will be needed to completely exchange one gram of zeolite that has one meq per gram of exchange capacity with two acetate anions to be exchanged (i.e. 183.4/1000/2=0.092).

Moreover, the exchange reactions of the present invention can be carried out either in hydrous or in anhydrous systems. This offers a great advantage for the preparation of anhydrous zeolites containing divalent and polyvalent cations. The preparation of divalent or polyvalent metal zeolites by ion-exchange is usually carried out in an aqueous medium followed by their dehydration at elevated temperatures, during which many divalent metal zeolite cage structures collapse. The methodology of the present invention circumvents this problem and permits the preparation of anhydrous zeolites with divalent or polyvalent cations without requiring a high temperature dehydration step, since anhydrous forms of zeolites can now be exchanged with divalent or polyvalent cations in an anhydrous medium according to the teachings of the present invention.

In actual preparative process, a solution of zinc or titanium derivative in water or another solvent or solvent mixture is stirred with zeolite. Zinc zeolite is thus formed by the in-situ process, as shown in Equation 1, 2, and 3. Other divalent derivatives of zinc can also be used, such as zinc acetate, zinc carbonate, etc. in Equation 1 or 2. In addition to zinc, virtually any other monovalent, divalent, or polyvalent metal can be complexed with zeolite surface by such ion-pair bonds to prepare metal-zeolite ion-pairs. Examples include, but not limited to, titanium zeolite, copper zeolite, manganese zeolite, magnesium zeolite, calcium zeolite, iron zeolite, and such.

The UV absorbing efficacy of zinc and titanium zeolites of the present invention can be further boosted by the inclusion of certain organic UV absorbing compositions. Such UVA/UVB sunscreen composition can be selected from, but not limited to Galanga extract (Kaempferia galanga), Benzophenone-3, Benzophenone-4, Ethylhexyl Methoxycinnamate, Homosalate, Ethyl hexyl salicylate, Octocrylene, Menthyl anthranilate, Avobenzone, Lawsone, Sulisobenzone, Trolamine salicylate, Lawsone, Glyceryl aminobenzoate, Cinoxate, and PABA, and combinations thereof.

### EXAMPLES

The following examples are presented to illustrate presently preferred practice thereof. As illustrations they are not intended to limit the scope of the invention. All quantities are in weight %.

### Example 1

### Preparation of Zinc Zeolite from Zinc Chloride

(1) Zeolite, Type 4A 20.0 (2) Zinc chloride 1.36 (3) Glycerin 78.64. Procedure. Mix (2) and (3) to a clear solution. Add (1) and mix. The mixture contains Zinc zeolite (100% zeolite exchanged), made by the in-situ ion-pair exchange.

### Example 2

### Preparation of Zinc Zeolite from Zinc Acetate

(1) Zeolite, Type 4A 40.0 (2) Zinc Acetate 0.18 (3) Glycerin 59.82. Procedure. Mix (2) and (3) to a clear solution. Add (1) and mix. The mixture contains Zinc zeolite (5% zeolite exchanged), made by the in-situ ion-pair exchange.

### Example 3

### Sunscreen Fluid Composition

(1) PEG-6 85.4 (2) Vitamin A Palmitate 0.1 (3) Vitamin E Acetate 0.1 (4) Phenoxyethanol 0.5 (5) Propyl Paraben 0.3 (6) Shea butter 1.0 (7) Apricot Kernel Oil 0.5 (8) Grapeseed Oil 0.5 (9) Kiwi Fruit Seed Oil 0.5 (10) Mango butter 0.5 (11) Hydroxypropyl Cellulose 0.5 (12) Zinc Zeolite 10.0 (13) Esculoside 0.5 (14) Darutoside 0.5 (15) Vitamin K 0.1. Procedure. Mix all ingredients to a paste.

### Example 4

### UV Absorbing Butter Composition

(1) Grapeseed Oil 15.8 (2) Mango Butter 18.5 (3) Cocoa Butter 0.5 (4) Beeswax 1.0 (5) Aloe butter 0.2 (6) Avocado Butter 0.5 (7) Shea Butter 0.5 (8) Vitamin E 0.1 (9) Grapeseed Oil 2.0 (10) Dimethicone 1.0 (11) Hydrogenated Soybean Oil 35.0 (12) Sesame Oil 0.9 (13) Tinoguard TT 0.2 (14) Phenoxyethanol 0.5 (15) Propyl Paraben 0.2 (16) Zinc Zeolite 15.0 (17) Titanium Zeolite 2.0 (18) Esculoside 0.5 (19) Darutoside 0.5 (20) Vitamin K 0.1 (21) Corn starch 5.0. Procedure: Mix all ingredients and heat at 60 to 70 C. Cool to room temperature. A butter-like material is obtained.

### Example 5

### Sunscreen Emollient Paste

(1) Paraffin Wax 25.0 (2) Propyl Paraben 0.1 (3) Cetyl Alcohol 1.0 (4) GMS-SE 4.0 (5) Stearic Acid 3.0 (6) Polawax 5.0 (7) Deionized Water 44.0 (8) Methyl Paraben 0.2 (9) Aloe vera 0.2 (10) Triethanolamine 0.5 (11) Dimethicone/Dimethiconol 2.0 (12) Zinc Zeolite 10.0 (13) Titanium Zeolite 4.0 (14) Esculin 0.5 (15) Boswellia serrata 0.5. Procedure. Mix ingredients (1) to (11) and heat at 80 to 90 C to a uniform mixture. Cool to 40 to 50 C. Add all other ingredients and mix. Cool to room temperature. An off-white paste is obtained.

### Example 6

### Sunscreen Powder

(1) Corn Starch 70.0 (2) Zinc Zeolite 14.0 (3) PEG-6 5.0 (4) Titanium Zeolite 5.0 (5) Tetrahydrocurcumin 0.5 (6) Vitamin K-1 0.5 (7) Dimethicone 5.0. Procedure. Mix (1) and (2). Premix (3) to (6) and add to main batch and mix. A powder composition is obtained.

1. A UV absorbing and free radical neutralizing composition comprising;
(i) At least one divalent or polyvalent metal zeolite, and
(ii) A cosmetically or pharmaceutically acceptable delivery system or a carrier base composition.

2. A composition according to claim 1, wherein divalent or polyvalent metal zeolite is selected from zinc zeolite, titanium zeolite, or a combination thereof.

3. A composition according to claim 1, wherein divalent or polyvalent metal zeolite is made by an in-situ process from a metal derivative and an aluminosilicate zeolite composition.

4. A composition according to claim 1, wherein zeolite composition is selected from a group of aluminosilicates that can be either in hydrated or anhydrous forms.

5. A composition according to claim 1, wherein metal zeolite composition is a topical sunscreen composition.

6. A composition according to claim 1, wherein metal zeolite composition is a UV absorber composition.

7. A composition according to claim 1, wherein metal zeolite composition is a Free radical neutralizing composition.

8. A composition according to claim 1, wherein a cosmetically acceptable delivery system or a carrier base is selected in the form of a lotion, cream, gel, spray, thin liquid, body splash, mask, serum, solid cosmetic stick, lip balm, shampoo, liquid soap, bar soap, bath oil, paste, salve, powder, collodion, impregnated patch, impregnated strip, skin surface implant, diaper, and any other such cosmetically or pharmaceutically acceptable topical delivery forms.

9. The compositions according to claim 1, wherein the cosmetically or pharmaceutically acceptable delivery system is traditional water and oil emulsions, suspensions, colloids, microemulsions, clear solutions, suspensions of nanoparticles, emulsions of nanoparticles, powders, or anhydrous compositions.

10. A composition according to claim 1, wherein cosmetically or pharmaceutically acceptable delivery system or carrier base optionally includes additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.
